(19) United States
(12) Patent Application Publication
(10) Pub. No.: US 2019/0307713 A1
(43) Pub. Date: Oct. 10, 2019

(54) COMBINATIONS OF DICLOFENAC AND H2 RECEPTOR ANTAGONISTS FOR THE TREATMENT OF PAIN AND INFLAMMATION

(71) Applicant: Mehmet Nevzat PISAK, Istanbul (TR)

(72) Inventor: Mehmet Nevzat PISAK, Istanbul (TR)

(21) Appl. No.: 16/465,862

(22) PCT Filed: Dec. 7, 2017
(86) PCT No.: PCT/TR2017/050637

§ 371 Date: May 31, 2019

(30) Foreign Application Priority Data
Dec. 7, 2016 (TR) 2016/18037
Aug. 9, 2017 (TR) 2017/11764
Nov. 2, 2017 (TR) 2017/17134

(51) Int. Cl. A61K 31/196; A61K 31/426; A61K 9/28; A61P 29/00

(52) U.S. Cl. CPC A61K 31/196; A61P 29/00; A61K 9/2886; A61K 31/426

(57) ABSTRACT

A single unit pharmaceutical dosage form comprising: diclofenac or a pharmaceutically acceptable salt thereof; and an H2 receptor antagonist, preferably famotidine or a pharmaceutically acceptable salt thereof, wherein diclofenac has a Tmax of between about and 50 minutes after administering said formulation to a patient.

### TECHNICAL FIELD

The present invention is related to the combined use of diclofenac and an H₂ receptor antagonist.

### BACKGROUND ART

It is well known that NSAIDs have the potential to cause gastrointestinal (GI) side effects, such as gastric and duodenal ulcers, bleeding and perforation, esophageal inflammation and strictures, and small bowel and colonic ulcers and strictures.

Laine L. (Semin. Arthritis Rheumatism. 2002; 32:25-32) reports that NSAIDs exert their pharmacological action by inhibiting the synthesis of prostaglandins (PGs) by non-selectively blocking cyclooxygenases 1 and 2 (COX-1 and COX-2) or by selectively blocking COX-2. Inhibition of COX-1 is also responsible, in part, for gastrointestinal side effects, which are the most frequent side effects of NSAIDs.

Gwaltney-Brant S. M. reports that non-selective COX inhibitors have other contributors to their gastrointestinal side effects, which are the carboxylic acid group in compounds, such as aspirin, ibuprofen and diclofenac, and the acidic enolic group in oxicams, such as piroxicam (Charlene A. M., editor. Comprehensive Toxicology. 2nd ed. Elsevier; Oxford, UK: 2010. pp. 159-161). These acidic groups cause local irritation upon oral administration, which can lead to the clinically observed gastrointestinal side effects either independently or in tandem with inhibition of the COX-1 enzyme.

In a report, Lanas et al. (2011) have concluded that more than 90% of the treated patients with osteoarthritis are at increased GI risk, with 60% of them at high risk. Diclofenac is a proven, commonly prescribed NSAID that has analgesic, anti-inflammatory, and antipyretic properties, and has been shown to be effective in treating a variety of acute and chronic pain and inflammatory conditions.

Vane J R. et al. (Nat N Biol. 1971; 231(25):232-5), Ku E C. et al. (Am J Med. 1986; 80(4B):18-23), and Patrono C. et al. (J Clin Invest. 2001; 108(1):7-13) report that diclofenac belongs to a group of NSAIDs that inhibit both COX-1 and COX-2 enzymes. The binding of NSAIDs to COX isozymes inhibits the synthesis of prostanoids (i.e., prostaglandin [PG]-E2, PGD2, PGF2, prostacyclin [PGI2], and thromboxane [TX] A2).

Furthermore, Patrono C. et al. (J Clin Invest. 2001; 108(1):7-13), Smyth E M et al., and Grosser T. et al. (J Clin Invest 2006; 116(44-15) report that PGE2 is the dominant prostanoid produced in inflammation, and the inhibition of its synthesis by NSAIDs is believed to be the main mechanism of the potent analgesic and anti-inflammatory properties of these agents.

Diclofenac, similar to other NSAIDs, is associated with an increased risk of serious dose-related GI side effects. Since its introduction in 1973, a number of different diclofenac-containing drug products have been developed with the goal of improving efficacy, tolerability, and patient convenience.

Another problem associated with diclofenac is that, when it is orally administered by means of instant or immediate release formulations, the corresponding Tmax (the time to the maximum plasma concentration) is usually located at about 1 hour since administration, this being of course a not completely satisfactory result when a prompt and strong analgesic/anti-pyretic effect is sought for. Attempts are therefore still being made in order to enhance the rate of absorption of diclofenac and to provide an earlier onset of the therapeutic effect.

Due to the fact that NSAIDs can cause GI ulceration, bleeding and perforation, especially in case of high dose usage, long-term treatments, history of a gastrointestinal disease or sensitivity to develop disease, additional use of gastro protective agent drugs for reducing GI side effects is needed. There have been binary combinations of NSAIDs and gastro protective agents in single unit dosage forms disclosed in the state of the art, none of which have disclosed diclofenac in combination with famotidine providing a faster onset of pain relief, faster anti-inflammatory action or faster anti-pyretic effect.

European patent EP 1411900 B1 relates to a multi-layer tablet suitable for oral administration comprising an outer layer of an acid inhibitor (H₂-receptor antagonist, preferably famotidine, or proton pump inhibitor), an inner core of an NSAID (preferably aspirin or naproxen) and a barrier coating surrounding the inner core of said NSAID, which might create manufacturing difficulties, stability issues.

US patent application US 20090233970 A1 relates to co-administration of a NSAID and acid blocking agent for the treatment of pain and inflammation with reduced gastrointestinal irritation. The application discloses that the two components can be administered in the same dosage form as a pharmaceutical composition comprising at least one NSAID (preferably naproxen or ibuprofen); and at least one acid blocking agent (preferably ranitidine).

To reduce the risk of serious GI adverse effects associated with long-term use of diclofenac sodium, combination therapies based binary combinations of diclofenac with gastroprotective agents such as prostaglandin analogs or proton pump inhibitors were developed.

US patent application US 20070237820 A1 relates to a solid oral dosage form comprising a first portion comprising an NSAID (preferably diclofenac); and a coating comprising an anti-ulcerative compound (preferably misoprostol); said coating at least partially surrounding said first NSAID portion. The pharmaceutical dosage described therein creates manufacturing difficulties.

Aim of the present invention is to provide a single unit pharmaceutical dosage form comprising at least one NSAID, preferably an NSAID that is COX-1 and COX-2 inhibitor and at least one H₂ receptor antagonist as a gastro protective agent.

Another aim of the present invention is to provide a single unit pharmaceutical dosage form of an NSAID, preferably a NSAID that is COX-1 and COX-2 inhibitor in combination with an H₂ receptor antagonist as a gastro protective agent, having a three-fold advantage in a synergistic manner decreasing the side effects while inducing an increase in the blood concentration of the NSAID in a shorter time compared to the mono administration of the NSAID.

### BRIEF DESCRIPTION OF THE INVENTION

The present invention provides a single unit pharmaceutical dosage form comprising diclofenac or a pharmaceutically acceptable salt thereof and one or more H₂ receptor antagonists or a pharmaceutically acceptable salt thereof.

The pharmaceutical dosage form of the present invention is synergistically useful for the treatment of inflammation and/or pain by reducing gastrointestinal side effects, increasing the blood concentration of diclofenac and shortening Tmax of diclofenac.

The present invention also provides a method for obtaining a Tmax of diclofenac in a human patient between about 5 to 40 minutes after administrating said diclofenac to said patient, comprising orally administering a diclofenac formulation to said patient, wherein said diclofenac formulation comprises diclofenac in acid and/or salt form together with one or more H₂ receptor antagonists, and one or more pharmaceutically acceptable excipients. Preferably, the method of the present invention provides a Tmax of diclofenac between about 5 to 30 minutes without decreasing the Cmax of diclofenac.

### DETAILED DESCRIPTION OF THE INVENTION

The present invention provides a single unit pharmaceutical dosage form comprising diclofenac as an NSAID and at least one H₂ receptor antagonist as a gastro protective agent. The single unit pharmaceutical dosage form of the present invention is useful for a faster onset of pain relief or antipyretic/anti-inflammatory effect, with decreased GI side effects.

When diclofenac is administered in combination with an H₂ receptor antagonist, the efficacy of the pain treatment has increased due to increased patient compliance caused by the diminished side effects and increased permeability and consequent absorption of diclofenac.

The combined use of diclofenac together with famotidine yields diclofenac based pharmaceutical compositions in which the active ingredient is absorbed more rapidly compared with formulations comprising diclofenac as a single active compound, bringing about higher blood levels and therefore a more immediate therapeutic effect. Accordingly, the combined use of diclofenac together with famotidine, when it is orally administered by means of instant or immediate release formulations the corresponding Tmax is shorter compared with the mono administration of diclofenac.

These surprising effects provide a three-fold advantage to diclofenac based pharmaceutical compositions for oral use, that GI side effects associated with the use of diclofenac decreases, blood concentration of diclofenac increases and Tmax becomes shorter compared to the mono administration of diclofenac. This will create a new treatment modality when a faster onset of action is needed without the unwanted GI side effects.

For the purposes of the present invention Tmax means the time to the maximum plasma concentration whereas Cmax is the maximum plasma concentration of the active ingredient.

Diclofenac which is the NSAID used in the present invention is a COX-1 and COX-2 inhibitor. Other NSAIDs do not necessarily share the same mechanism of action. Diclofenac can also inhibit the thromboxane prostanoid receptor. Diclofenac is used in the treatment of inflammation and pain. It possesses analgesic and antipyretic properties as well as anti-inflammatory activity. Its chemical structure is shown in Formula 1.

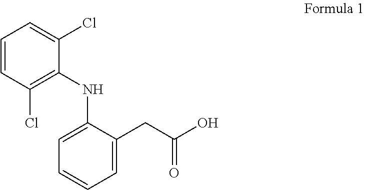 [SMILES: O=C(O)Cc1ccccc1Nc1c(Cl)cccc1Cl]

The chemical name of diclofenac is 2-[2-(2,6-dichloroanilino)phenyl]acetic acid.

In one embodiment, diclofenac is in the form of a free acid or in the form of a salt.

In one embodiment of the present invention, diclofenac is diclofenac sodium salt or diclofenac potassium salt.

Diclofenac potassium salt is associated with a faster absorption and consequentially a more rapid onset of pain relief compared with the sodium salt. Although it has a solid efficacy profile, the ulcerogenic properties and gastro intestinal side effects are, to a large extent related to diclofenac's inhibition of COX-1 in the gastric mucosa.

In the preferred embodiment of the present invention, diclofenac is diclofenac potassium.

In one embodiment of the present invention, H₂ receptor antagonist is famotidine, pibutidine, lafutidine, loxtidine, nizatidine, roxatidine, tiotidine, niperotidine, oxmetidine, or pharmaceutically acceptable salt thereof.

In another embodiment of the present invention, H₂ receptor antagonist is famotidine, or pharmaceutically acceptable salt thereof.

Famotidine is an H₂-receptor antagonist used in the treatment of gastrointestinal diseases. It protects the gastric mucosa against irritation. Its chemical structure is shown in Formula 2.

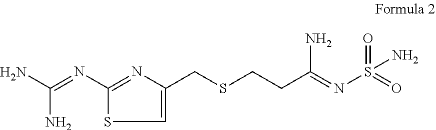 [SMILES: NC(N)=Nc1nc(CSCC/C(N)=N/S(N)(=O)=O)cs1]

The chemical name of famotidine is 3-(2-(diaminomcthyleneamino)thiazol-4-yl methylthio)-N-sulfamoylpropionamidine.

Famotidine has an ideal safety/efficacy ratio compared to the other gastro protective agents, when compared with the long term side effects created by proton pump inhibitors. Famotidine also provides a superior treatment to current therapies as it limits the gastro-intestinal side effects that will happen in a prophylactic manner, therefore creating fewer complications from efficient NSAID use in a long term.

The pharmaceutical dosage form of the present invention may comprise active components in the form of a racemic mixture, or in the form of substantially pure enantiomers or salts of enantiomers thereof.

The single unit pharmaceutical dosage form comprising diclofenac in its acid and/or salt form, and famotidine as a gastro protective agent provides an even faster onset of pain relief or antipyretic/anti-inflammatory effect, with decreased GI side effects, due to a shorter Tmax and higher Cmax.

In one embodiment of the present invention the single unit pharmaceutical dosage form comprising diclofenac and at least one H₂ receptor antagonist is provided for use in a method for the treatment of inflammation or pain with reduced GI side effects.

In one embodiment, the single unit pharmaceutical dosage form of the present invention is useful for the treatment of inflammation or pain caused by muscular or skeletal system disease.

In one embodiment, the single unit pharmaceutical dosage forms of the present invention is useful for the treatment of inflammation or pain caused by chronic polyarthritis, ankylosing spondilytis, osteoarthritis, gout attacks, extra-articular rheumatism, post-traumatic and postoperative pain, and dysmenorrhea.

In another embodiment, the single unit pharmaceutical dosage form of the present invention is useful as an antipyretic agent.

Suitable dosages of diclofenac as an NSAID and H₂ receptor antagonist as a gastro protective agent in single unit dosage form of the present invention can be selected according to the condition to be treated, as well as to provide synergistic effects in terms of faster onset of action and reduced gastrointestinal irritation.

In one embodiment of the present invention, the single unit pharmaceutical dosage form comprises diclofenac in an amount from about 12.5 to 225 mg.

In another embodiment, the single unit pharmaceutical dosage form of the present invention comprises diclofenac in an amount from about 12.5 to 100 mg.

In another embodiment the single unit pharmaceutical dosage form of the present invention comprises diclofenac in an amount of 25 to 100 mg.

In another embodiment the single unit pharmaceutical dosage form of the present invention comprises diclofenac in an amount from about 50 to 100 mg.

In another embodiment the single unit pharmaceutical dosage form of the present invention comprises diclofenac in an amount of 12.5 mg, 25 mg or 50 mg.

In one embodiment of the present invention, the single unit pharmaceutical dosage form comprises H₂ receptor antagonist in an amount from about 5 to 80 mg.

In another embodiment the single unit pharmaceutical dosage form of the present invention comprises H₂ receptor antagonist in an amount from about 10 to 60 mg.

In one embodiment of the present invention, the single unit pharmaceutical dosage form comprises H₂ receptor antagonist in an amount from about 20 to 40 mg.

In another embodiment the single unit pharmaceutical dosage form of the present invention comprises H₂ receptor antagonist in an amount of 20 or 40 mg.

In one embodiment, the single unit pharmaceutical dosage form of the present invention comprises diclofenac potassium together with famotidine and pharmaceutically acceptable excipients, in which formulation famotidine is present in an amount of from 10 to 90% by weight based on the weight of diclofenac.

As it will be clear from the examples, the single unit pharmaceutical dosage forms of the present invention comprising the administration of diclofenac potassium and famotidine combination creates a synergistic effect and attained it's Tmax and Cmax in 0.083 hours (approximately 5 minutes) at 1.2625 nanogram/ml compared to the administration of diclofenac potassium alone which attained 1.2435 nanogram/ml at 0.25 hours. Thus the composition of the present invention attained a surprising 3 times faster Tmax with a higher Cmax. Accordingly claimed diclofenac based formulations permit to achieve a higher Cmax in a shorter Tmax with less GI side effects. Additionally, these formulations have a more rapid onset of action than the formulations where the effect of diclofenac for the treatment of pain and/or inflammation begins in about 20 minutes.

In one embodiment of the present invention the single unit pharmaceutical dosage form of the present invention has a Tmax of between 5 to 50 minutes after administering said formulation to a patient.

In another embodiment, the single unit pharmaceutical dosage form of the present invention has a Tmax of between 5 to 40 minutes after administering said formulation to a patient.

In another embodiment, the single unit pharmaceutical dosage form of the present invention has a Tmax of between 5 to 30 minutes after administering said formulation to a patient. In another embodiment, the single unit pharmaceutical dosage form of the present invention has a Tmax of between 10 to 30 minutes after administering said formulation to a patient.

In another embodiment, the single unit pharmaceutical dosage form of the present invention has a Tmax of at least 2 times faster compared to diclofenac alone.

In another embodiment, the single unit pharmaceutical dosage form of the present invention has a Tmax of at least 10% faster compared to diclofenac alone.

In another embodiment, the single unit pharmaceutical dosage form comprising diclofenac and famotidine wherein famotidine increases the permeability and absorption rate of diclofenac by at least 3%.

In one embodiment of the present invention the single unit pharmaceutical dosage form of the present invention is in tablet, capsule or powder form, for a faster onset of action. It is important for the tablets or capsules to have an instant or immediate release profile in the gastrointestinal system. Powder form can be a pack of powder for dissolving or suspending in water.

In one embodiment, an oral single unit pharmaceutical dosage form of the present invention has an instant or immediate release profile, with at least 25% of diclofenac and at least 25% of H₂ receptor antagonist released before reaching the intestines.

In another embodiment at least 6.25 mg of diclofenac is released before reaching the intestines.

In another embodiment at least 12.5 mg of diclofenac is released before reaching the intestines.

In another embodiment at least 5 mg of H₂ receptor antagonist is released before reaching the intestines.

In one embodiment, at least 25% of the active ingredients, most preferably diclofenac is released in the intestines and H₂ receptor antagonist has an instant or immediate release profile.

In one embodiment, at least 60% of the active ingredients, most preferably diclofenac is released in the intestines and H₂ receptor antagonist has a normal or immediate release profile.

In one embodiment, an oral single unit pharmaceutical dosage of the present invention has a sustained or modified release profile, with at least 70% of diclofenac released in the intestines.

In one embodiment, an oral single unit pharmaceutical dosage of the present invention has a sustained or modified release profile, with at least 35 mg of diclofenac released in the intestines.

In another embodiment, an oral single unit pharmaceutical dosage of the present invention has a sustained or modified release profile, with at least 52.5 mg of diclofenac released in the intestines.

In one embodiment, an oral single unit pharmaceutical dosage of the present invention has a sustained or modified release profile, with at least 70 mg of diclofenac released in the intestines.

In accordance with the above embodiments, a ratio of diclofenac to the H₂ receptor antagonist is from about 1.25 to 1, from about 2.5 to 1 or from about 3.75 to 1.

In accordance with the above embodiments, a ratio of diclofenac to the H₂ receptor antagonist is from about 0.625 to 1, from about 1.25 to 1, from about 1.875 to 1 and from about 2.5 to 1 by weight.

The fact that famotidine creates this synergistic effect when coupled with diclofenac has a threefold advantage which has never attained before; 1: Higher concentration of diclofenac in blood, 2: in a shorter period of time compared with the traditional diclofenac potassium tablets or capsules, 3: protection of the gastric mucosa due to the gastroprotective properties of famotidine, therefore creating the perfect NSAID treatment modality when a fast onset of action is needed, especially for patients that have GI related problems.

The single unit pharmaceutical dosage form of the present invention may be an oral dosage form. This oral dosage form may be tablet, capsule, pellet, granule, tablet in tablet, tablet in capsule, powder or in coated tablet form, preferably being in tablet, capsule or powder form. The usual initial dose of diclofenac in the single unit pharmaceutical dosage form is 12.5 to 100 mg by oral administration in the form of tablet, capsule, dragee or powder form. It is also used for intramuscular and intravenous administration or for topical application as gel, suppository or ophthalmic preparation. The usual initial dose of famotidine in the single unit pharmaceutical dosage form is 20 to 40 mg by oral administration in the form of tablet, capsule and suspension or intravenous administration.

In one embodiment of the present invention the single unit pharmaceutical dosage form of the present invention is in the form of an instant release tablet.

In one embodiment of the present invention the single unit pharmaceutical dosage form of the present invention is in the form of an instant release capsule.

Dosage forms of the drug may be administered once, twice or thrice a day.

In one embodiment a single unit pharmaceutical dosage form of the present invention further comprises at least one pharmaceutically acceptable excipient such as a carrier.

Oral dosage forms of the present invention may comprise suitable diluents, binders, lubricants, disintegrating agents, surfactants, sweetening agents, coloring agents and coating agents.

Pharmaceutically acceptable diluents of the present invention may be selected from magnesium stearate, lactose, microcrystalline cellulose, starch, pre-gelatinized starch, calcium phosphate, calcium sulfate, calcium carbonate, mannitol, sorbitol, xylitol, sucrose, maltose, fructose, dextrose and the like. Pharmaceutically acceptable binders of the invention may be selected from starches, natural sugars, corn sweeteners, natural and synthetic gums, cellulose derivatives, gelatin, PVP, polyethylene glycol, waxes, sodium alginate, alcohols, water and the like.

Pharmaceutically acceptable lubricants of the present invention may be selected from metallic stearates, metallic lauryl sulfates, fatty acids, fatty acid esters, fatty alcohols, paraffins, hydrogenated vegetable oils, polyethylene glycols, boric acid, sodium benzoate, sodium acetate, sodium chloride, talc and the like.

Pharmaceutically acceptable disintegrating agents of the present invention may be selected from starches, cellulose derivatives, PVP, crospovidone, clays, ion-exchange resins, alginic acid, sodium alginate and the like.

Pharmaceutically acceptable surfactants of the present invention may be selected from sulfates, sulfonates, phosphates, carboxylates, primary-secondary-tertiary amines, quaternary ammonium compounds, fatty alcohols, sugar esters of fatty acids, glycerides of fatty acids, polyoxy ethylene glycol alkyl ethers, polisorbates, sorbitan alkyl esters, poloxamers and the like.

It will be evident to any skilled in this art that the present formulations can also be used as instant or immediate release layers of multilayered release pharmaceutical formulations containing diclofenac and at least one H₂ receptor antagonist, preferably famotidine as active ingredients; said formulations are therefore a further object of the present invention.

A process for the manufacture of a single unit pharmaceutical dosage form of the present invention comprises the steps of:
i) dissolving a certain amount of of at least one pharmaceutically acceptable excipient in purified water,
ii) mixing diclofenac, and at least one pharmaceutically acceptable excipient together; iii) wet-granulating the obtained mixture with the excipient solution;
iv) drying the granules so-obtained and passing the granules through the dry granulator;
v) adding H₂ receptor antagonist to the granules obtained after the step (iv);
vi) applying suitable mixing,
vii) obtaining final mixture by the addition of at least one pharmaceutically acceptable excipient to the mixture; and
viii) pressing and film-coating the tablets.

A process for the manufacture of a single unit pharmaceutical dosage form of the present invention comprises the steps of:
i) dissolving a certain amount of at least one pharmaceutically acceptable excipient in purified water,
ii) mixing diclofenac and H₂ receptor antagonist, and at least one pharmaceutically acceptable excipient together;
iii) wet-granulating the obtained mixture with the excipient solution;
iv) drying the granules so-obtained and passing the granules through the dry granulator;
v) obtaining final mixture by the addition of at least one pharmaceutically acceptable excipient to the mixture; and
vi) pressing and film-coating the tablets.

A process for the manufacture of a single unit pharmaceutical dosage form of the present invention comprises the steps of:
i) providing diclofenac together with neutral cores and optionally at least one pharmaceutically acceptable excipient present in the core portion, wherein the core portion is obtained by spraying the solution comprising diclofenac onto the neutral cores;
ii) applying enteric coating to protect the diclofenac pellets against gastric acid for a sustained release effect;
iii) providing H₂ receptor antagonist together with neutral cores and optionally at least one pharmaceutically acceptable excipient present in the core portion wherein the core portion is obtained by spraying the solution comprising H₂ receptor antagonist onto the neutral cores;
iv) applying enteric coating to protect the intermediate-coated pellets against gastric acid; and
v) filling the coated diclofenac potassium pellets obtained as a result of the step ii) and immediate or sustained release H₂ receptor antagonist pellets obtained as a result of the step iii) into the capsules.

A process for the manufacture of a single unit pharmaceutical dosage form of the present invention comprises the steps of:
i) providing diclofenac together with neutral cores and optionally at least one pharmaceutically acceptable excipient present in the core portion, wherein the core portion is obtained by spraying the solution comprising diclofenac onto the neutral cores;
ii) applying enteric coating to protect the diclofenac pellets against gastric acid for a sustained release effect;
iii) providing H₂ receptor antagonist together with neutral cores and optionally at least one pharmaceutically acceptable excipient present in the core portion wherein the core portion is obtained by spraying the solution comprising H₂ receptor antagonist onto the neutral cores;
iv) applying enteric coating to protect the intermediate-coated pellets against gastric acid;
v) filling the coated diclofenac potassium pellets obtained as a result of the step ii) and immediate or sustained release H₂ receptor antagonist pellets obtained as a result of the step iii) into the capsules; and
vi) coating the core portion with an intermediate coating which separates the core portion from outer coating and which contributes to stability.

### EXAMPLES

The present invention will be explained more in detail by illustrating Examples and studies.

### Example 1

Table 1 provides the contents of an embodiment of the present invention. The composition is in the form of a film coated tablet of diclofenac potassium in combination with famotidine.

TABLE 1
Film coated tablet of diclofenac potassium
in combination with famotidine.

| Illustrative Film Tablet | Weight (mg)/unit dose |
|---|---|
| Diclofenac potassium | 50 |
| Famotidine | 20 |
| Lactose granule | 64 |
| Microcrystalline cellulose | 67 |
| Hydroxypropyl Cellulose | 8 |
| Crospovidone | 10 |
| Colloidal silicon dioxide | 2 |
| Magnesium stearate | 2 |
| Coating based on copovidone with | 7 |
| cellulosic polymers | |

### Example 2

Preparation methods of the pharmaceutical compositions of the inventions

1. Preparation Method Wherein the Active Components are not Directly Mixed:

At the first stage, a certain amount of PVP (K-30) is dissolved in purified water. Diclofenac, remaining amount of PVP (K-30), lactose, microcrystalline cellulose, sodium starch glycolate and colloidal silicon dioxide are mixed together. The obtained mixture is wet-granulated with the PVP (K-30) solution. Granules so-obtained are dried and pass through the dry granulator.

At the second stage, famotidine is added to the granules obtained in the first stage. Suitable mixing is applied. Final mixture is obtained by the addition of magnesium stearate to the mixture. After the tablets are pressed, they are film-coated.

2. Preparation Method Wherein the Active Components are Directly Mixed:

A certain amount of PVP (K-30) is dissolved in purified water. Diclofenac, famotidine, remaining amount of PVP (K-30), lactose, microcrystalline cellulose, sodium starch glycolate and colloidal silicon dioxide are mixed together. The obtained mixture is wet-granulated with the PVP (K-30) solution. Granules so-obtained are dried and pass through the dry granulator. Final mixture is obtained by the addition of magnesium stearate to the mixture. After the tablets are pressed, they are film-coated.

Example for a faster onset of action and pain relief with less GI side effects:

### Example 3

Table 2 below provides the contents of an example composition in the form of a film coated tablet of diclofenac in combination with famotidine.

TABLE 2
Film Tablet Composition

| Illustrative Film Tablet | Weight (mg)/unit dose |
|---|---|
| Core | |
| Diclofenac potassium or sodium | 25.00 to 100.00 |
| Famotidine | 20.00 or 40.00 |
| Lactose | 50 to 240.00 |
| Microcrystalline cellulose | 30 to 142.00 |
| Sodium starch glycolate | 20 to 66.00 |
| Colloidal silicon dioxide | 05 to 2.80 |
| PVP (K-30) | 7 to 38.40 |
| Magnesium stearate | 3 to 9.80 |
| Film Coating | |
| Hidroxypropylmethyl cellulose | 7 to 35.40 |
| Titanium dioxide | 4 to 16.70 |
| PEG 400 | 0.5 to 3.90 |

### Example 4

Preparation methods of the pharmaceutical compositions of the inventions

1. Preparation Method Wherein the Active Components are not Directly Mixed:

At the first stage, a certain amount of PVP (K-30) is dissolved in purified water. Diclofenac, remaining amount of PVP (K-30), lactose, microcrystalline cellulose, sodium starch glycolate and colloidal silicon dioxide are mixed together. The obtained mixture is wet-granulated with the PVP (K-30) solution. Granules so-obtained are dried and pass through the dry granulator.

At the second stage, famotidine is added to the granules obtained in the first stage. Suitable mixing is applied. Final mixture is obtained by the addition of magnesium stearate to the mixture. After the tablets are pressed, they are film-coated.

2. Preparation Method Wherein the Active Components are Directly Mixed:

A certain amount of PVP (K-30) is dissolved in purified water, diclofenac, famotidine, remaining amount of PVP (K-30), lactose, microcrystalline cellulose, sodium starch glycolate and colloidal silicon dioxide are mixed together. The obtained mixture is wet-granulated with the PVP (K-30) solution. Granules so-obtained are dried and pass through the dry granulator. Final mixture is obtained by the addition of magnesium stearate to the mixture. After the tablets are pressed, they are film-coated.

### Example 5

Oral dosage forms of this embodiment of the invention are preferably in capsule form:

Preparation method of the capsules of the invention has four stages. Diclofenac sodium or potassium pellets are obtained in the first stage; famotidine pellets are obtained in the second stage. In the third stage; diclofenac sodium or potassium pellets consisting of a core and an enteric coating surrounding the core and famotidine pellets consisting of a core, optionally an intermediate coating surrounding the core, and optionally an enteric coating surrounding the intermediate-coated pellets are obtained and filled into a capsule.

In the first stage, diclofenac sodium or potassium together with neutral cores and optionally at least one excipient selected from the pharmaceutically acceptable diluents, binders, lubricants, disintegrating agents and surfactants is present in the core portion. Pharmaceutically acceptable binders used in the core portion may preferably be selected from starches, natural sugars, cellulose derivatives, PVP, polyethylene glycol, alcohols and water. Said binder of the invention is more preferably a mixture of HPMC and water. The core portion is obtained by spraying the solution comprising diclofenac potassium onto the neutral cores.

As a continuation of the first stage, enteric coating is applied to protect the pellets in the first stage against gastric acid. Enteric coating polymers used in the enteric coating portion may be selected from methacrylic acid copolymers, cellulose acetate phthalate, cellulose acetate trimellitate, polyvinyl acetate phthalate, hydroxypropyl methylcellulose phthalate, hydroxypropyl methylcellulose acetate succinate, and the like. In addition, a plasticizer selected from phthalates, trimellitates, adipates, sebacates, maleates, benzoates, sulfonamides, organophosphates, glycols/polyethers, acetylated monoglycerides, alkyl citrates, polysorbates, triacetine, cetyl alcohol, and the like may also be used to improve the properties of coating such as elasticity, hardness and adhesion. Besides the polymer and plasticizer; a pure solvent or a solvent mixture and optionally at least one excipient selected from the pharmaceutically acceptable diluents, binders, lubricants, disintegrating agents and surfactants may also be added to prepare enteric coating solution. Enteric-coated pellets are obtained by spraying enteric coating solution onto the pellets of the core portion. Following the addition of talk, enteric-coated pellets so-obtained are dried at 40° C.; and said enteric-coated pellets are ready for capsule fill.

In the second stage, famotidine together with neutral cores and optionally at least one excipient selected from the pharmaceutically acceptable diluents, binders, lubricants, disintegrating agents and surfactants is present in the core portion. Pharmaceutically acceptable binders used in the core portion may preferably be selected from starches, natural sugars, cellulose derivatives, PVP, polyethylene glycol, alcohols and water. Said binder of the invention is more preferably a mixture of HPMC and water. The core portion is obtained by spraying the solution comprising famotidine onto the neutral cores.

The core portion in the first or second stage is optionally coated with an intermediate coating. Intermediate coating is a portion that separates the core portion from outer coating and that contributes to provide stability. Pharmaceutically acceptable binders used in the intermediate coating portion may preferably be selected from starches, natural sugars, cellulose derivatives, PVP, polyethylene glycol, alcohols and water. Said binder of the invention is more preferably a mixture of HPMC, polyethylene glycol, ethanol and water. Optionally at least one excipient selected from the pharmaceutically acceptable diluents, lubricants, disintegrating agents and surfactants may also be added into this portion. Intermediate-coated pellets are obtained by spraying said suspension onto the core portion.

Enteric coating can be applied to protect the intermediate-coated pellets in the second stage against gastric acid. Enteric coating polymers used in the enteric coating portion may be selected from methacrylic acid copolymers, cellulose acetate phthalate, cellulose acetate trimellitate, polyvinyl acetate phthalate, hydroxypropyl methylcellulose phthalate, hydroxypropyl methylcellulose acetate succinate, and the like. In addition, a plasticizer selected from phthalates, trimellitates, adipates, sebacates, maleates, benzoates, sulfonamides, organophosphates, glycols/polyethers, acetylated monoglycerides, alkyl citrates, polysorbates, triacetine, cetyl alcohol, and the like may also be used to improve the properties of coating such as elasticity, hardness and adhesion. Besides the polymer and plasticizer; a pure solvent or a solvent mixture and optionally at least one excipient selected from the pharmaceutically acceptable diluents, binders, lubricants, disintegrating agents and surfactants may also be added to prepare enteric coating solution. Enteric-coated pellets are obtained by spraying enteric coating solution onto the intermediate-coated pellets. Enteric-coated pellets so-obtained are optionally mixed with at least one excipient selected from the pharmaceutically acceptable diluents, binders, lubricants, disintegrating agents and surfactants; and said enteric-pellets are ready for capsule fill.

Finally, in the third stage enteric-coated diclofenac potassium pellets obtained in the first stage; and immediate or sustained release famotidine pellets obtained in the third stage are filled into the capsules together.

### Example 6

Experimental Procedure to Compare the Pharmacokinetic Properties of Diclofenac Potassium Alone and Diclofenac Potassium in Combination with Famotidine

The primary objective of this experimental study was to observe if famotidine changed the pharmacokinetic properties of diclofenac potassium when diclofenac potassium is given alone and in combination with famotidine.

Animals

Male Wistar rats (240-260 g) were maintained in an air conditioned quarters at a temperature of 22±2° C. and a relative humidity of 50±10%. Food and water were allowed ad libitum. The animals were acclimatized to the facilities for five days, and then fasted with free access to water for 12 h prior to the experiment. All these animals were housed under similar conditions.

Drug Administration

Bioavailability and pharmacokinetics of diclofenac were studied in two different groups; the first group was administered diclofenac potassium alone and the second group was administered a combination of diclofenac potassium and famotidine following an oral administration of 1 mg/kg diclofenac potassium, 0.4 mg/kg famotidine in different occasions. The ratio of diclofenac to famotidine was 1 to 0.4; equal to 50 mg diclofenac and 20 mg famotidine in human dosing.

Six male and/or female rats per group were lavaged with 1 mg/kg Diclofenac potassium and a combination of diclofenac potassium and famotidine. Blood (0.2 ml) was taken from the tail vein prior to administration of test substances (0 h) and blood was taken after 0.083, 0.167, 0.25, 0.5, 1, 2, 4, 8 and 12 h respectively.

Extraction of Blood Samples

Blood samples were collected in tubes containing %5 Nat-EDTA and kept on ice until dichloromethane was added and they were centrifuged at 7000×g for 5 min at 4° C. and supernatants were collected for HPLC analysis.

Results

The surprising results detailed below demonstrate the pharmacokinetic superiority of the diclofenac and famotidine combination especially when Tmax is concerned. Futhermore in a 12 hour period after the oral administration of the combination of the present invention, surprisingly there isn't a fast decrease of the blood concentration, instead the addition of famotidine creates therapeutically effective blood concentrations throughout the 12 hour period.

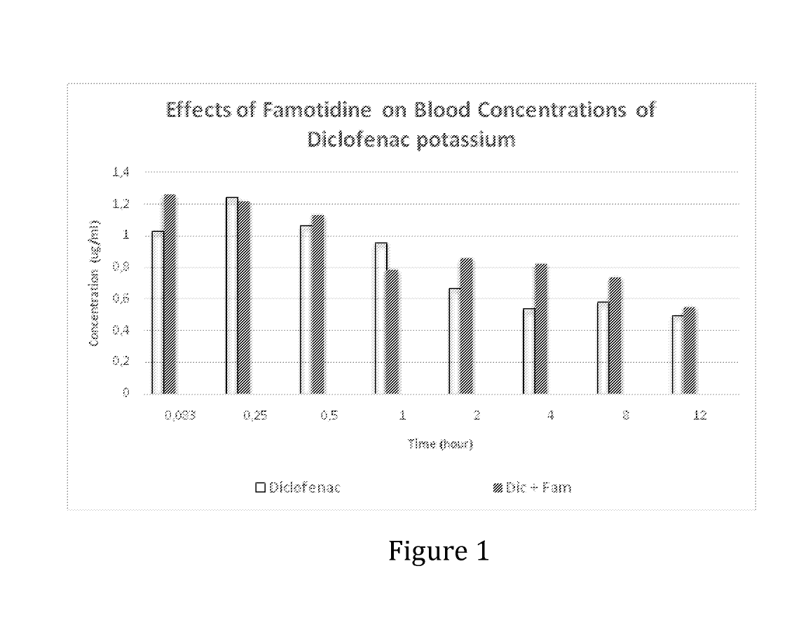

FIG. 1 shows the effects of famotidine on blood concentrations of diclofenac potassium.

Table 3 shows the blood concentrations of diclofenac potassium

TABLE 3
Blood Concentrations of Diclofenac Potassium

| | | | | | | | | |
|---|---|---|---|---|---|---|---|---|
| Time (h) | 0.083 | 0.25 | 0.5 | 1 | 2 | 4 | 8 | 12 |

Groups

| | | | | | | | | |
|---|---|---|---|---|---|---|---|---|
| Diclofenac | 1.031± | 1.2435± | 1.0615± | 0.9565± | 0.667± | 0.5355± | 0.584± | 0.499± |
| potassium | 0.0891 | 0.0346 | 0.0078 | 0.0361 | 0.0085 | 0.0177 | 0.0990 | 0.0877 |
| (ng/ml) | | | | | | | | |
| Diclofenac | 1.2625± | 1.223± | 1.137± | 0.788± | 0.86± | 0.8225± | 0.7395± | 0.551± |
| potassium + | 0.0771 | 0.0085 | 0.0156 | 0.2602 | 0.0099 | 0.0332 | 0.0064 | 0.0085 |
| Famotidine | | | | | | | | |
| (ng/ml) | | | | | | | | |

The results of the study demonstrate that pharmaceutical compositions of the present invention have superior pharmacokinetic profiles achieved when a shorter Tmax and faster onset of action is needed and that famotidine increases the Cmax of diclofenac.

### Example 7

Study on Permeability of the Active Ingredients Diclofenac Potassium and Famotidine Separately and in Combination Using Caco-2 Permeability Method

The permeability study designed using Caco-2 intestinal permeability model was prepared by using Millipore 96 well insert plate as 10,000 cells/well on Feb. 21, 2017 and placed in an incubator. Tests on integrity, viability and permeability were performed on Mar. 15, 2017.

Viability results were obtained by MTT and integrity results were obtained using Lucifer yellow and the plate was determined to be eligible for permeability study.

Application of Permeability Study

Diclofenac potassium MW: 334.237

Famotidine: 337.449

DMSO and methanol as solvent suitable for LC/MSMS analyses were tested and stock solutions of Diclofenac potassium in 50 mg/ml and Famotidine in 20 mg/ml DMSO were prepared and from these solutions, the samples were prepared in FaSSIF HBSS with HEPES to obtain final concentrations as 50 μg/ml for Diclofenac potassium and 20 μg/ml for Famotidine.

After the test was set, the samples were collected at 1 h, 1.5 h, 2 h, 4 h and 8 h and sample from basolateral was collected at 8 h to calculate coefficient of permeability.

Application of LC/MSMS Analyses

Methanol was used as solvent for the samples and the samples were analyzed in LC/MSMS 8040 analyzer using mobile phase, Methanol+bidistillated water+Ammonium formate.

Results

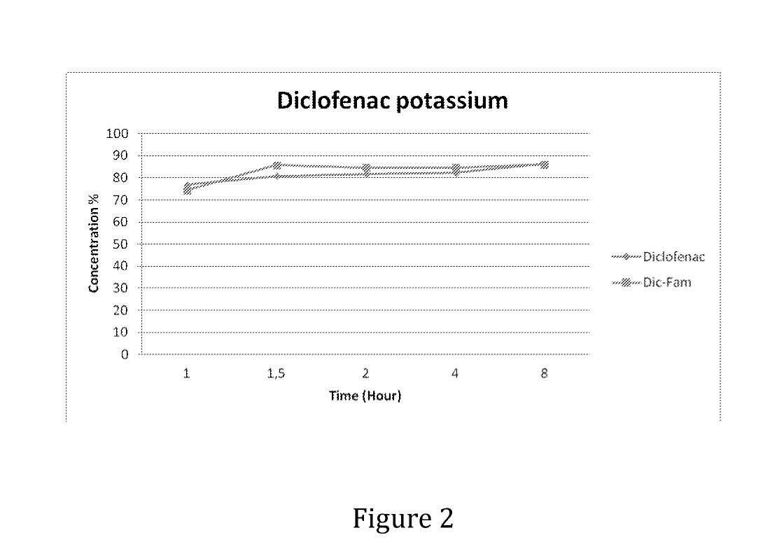

FIG. 2 shows intestinal permeability of diclofenac potassium separately and in combination with famotidine.

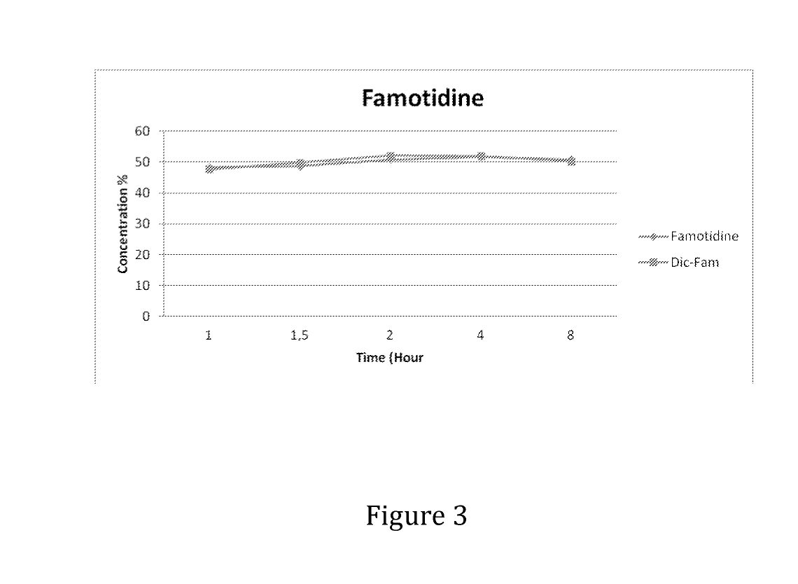

FIG. 3 shows intestinal permeability of famotidine separately and in combination with diclofenac potassium.

Table 4 shows the permeability percentages of diclofenac potassium separately and in combination with famotidine.

Table 5 shows the permeability percentages of famotidine separately and in combination with diclofenac potassium.

TABLE 4
Permeability percentages of Diclofenac potassium
separately and in combination with Famotidine.

| | 1 | 1.5 | 2 | 4 | 8 |
|---|---|---|---|---|---|
| | hour | hours | hours | hours | hours |
| Diclofenac potassium | 76.93 | 80.76 | 81.80 | 82.22 | 86.32 |
| Diclofenac potassium + | 74.38 | 85.66 | 84.63 | 84.64 | 86.05 |
| Famotidine | | | | | |

TABLE 5
Permeability percentages of Famotidine separately
and in combination with Diclofenac potassium.

| | 1 | 1.5 | 2 | 4 | 8 |
|---|---|---|---|---|---|
| | hour | hours | hours | hours | hours |
| Famotidine | 48.36 | 48.67 | 50.80 | 51.67 | 50.80 |
| Famotidine + Diclofenac | 47.64 | 49.79 | 52.08 | 51.95 | 50.23 |
| potassium | | | | | |

Evaluation of the Results

Diclofenac potassium is a BCS Class II medicinal product in the form of a weak acid. BCS Class II medicinal products are characterized by low solubility and high permeability. Diclofenac potassium alone showed 76.93% permeability at 1 h. Diclofenac potassium showed 80.76% permeability separately and 85.66% in combination with famotidine at 1.5 h, and Diclofenac potassium showed 81.80% permeability separately and 84.63% in combination with famotidine at 2 h. Percentages at 4 h and 8 h were found to be equivalent Based on the data obtained, Famotidine increases intestinal permeability of Diclofenac potassium by 9% at 1.5 h and 2 h. These data indicate that Famotidine increases the permeability and absorption rate of Diclofenac potassium by 9%, which was even more surprising considering that diclofenac is a BCS class II drug and already has high permeability, so to further increase this parameter was astonishing. Accordingly, permeability and absorption rate of Diclofenac potassium will increase in the presence of Famotidine under in-vivo conditions. It would be known by anyone skilled in the art, that the increase in the permeability and absorption of diclofenac does definitely increase the blood concentration of diclofenac in-vivo and this increase is created by the combination of famotidine with diclofenac.

These values are only evaluated for the active ingredients.

In conclusion, presence of Famotidine increases the intestinal permeability of and absorption rate of Diclofenac potassium by at least 3%.

1. An instant release single unit oral pharmaceutical dosage form for the treatment of inflammation or pain comprising:
a) diclofenac potassium in an amount of about 12.5 mg, 25 mg or 50 mg; and
b) famotidine, wherein the dosage form is obtained by a process comprising the step of wet granulating diclofenac and wherein diclofenac has tmax between 5 and 50 minutes after administering the dosage form.

2.-7. (canceled)

8. The single unit pharmaceutical dosage form according to claim 1, comprising famotidine in an amount from about 20 to 40 mg.

9. The single unit pharmaceutical dosage form according to claim 1, wherein famotidine is present in an amount of from 10 to 90% by weight based on the weight of diclofenac.

10. (canceled)

11. The single unit pharmaceutical dosage form according to anyone of claim 1, with an instant release profile, wherein at least 25% of diclofenac and at least 25% of H2 receptor antagonist are released before reaching the intestines.

12. The single unit pharmaceutical dosage form according to claim 11, wherein at least 6.25 mg of diclofenac is released before reaching the intestines.

13. The single unit pharmaceutical dosage form according to claim 11, wherein at least 5 mg of H2 receptor antagonist is released before reaching the intestines.

14. (canceled)

15. (canceled)

16. The single unit pharmaceutical dosage form according to anyone of claim 1 for use in the treatment of inflammation or pain with reduced gastrointestinal side effects.

17. The single unit pharmaceutical dosage form according to anyone of claim 1 for use in the treatment of pain or inflammation wherein inflammation or pain is caused by chronic polyarthritis, ankylosing spondilytis, osteoarthritis, gout attacks, extra-articular rheumatism, post-traumatic and postoperative pain, rheumatoid arthritis and dysmenorrhea.

18. A single unit pharmaceutical dosage form according to anyone of claim 1 wherein famotidine increases the permeability and absorption rate of diclofenac by at least 3%.

19. A process for the manufacture of a single unit pharmaceutical dosage form according to anyone of claim 1, wherein the process comprises the steps of:
i) dissolving a certain amount of at least one pharmaceutically acceptable excipient in purified water;
ii) mixing diclofenac potassium, and at least one pharmaceutically acceptable excipient together;
iii) wet-granulating the obtained mixture with the excipient solution;
iv) drying the granules so-obtained and passing the granules through the dry granulator;
v) adding famotidine to the granules obtained after the step (iv);
vi) applying suitable mixing,
vii) obtaining final mixture by the addition of at least one pharmaceutically acceptable excipient to the mixture; and
viii) pressing and film-coating the tablets.

20.-23. (canceled)
